(19) United States
(12) Patent Application Publication
(10) Pub. No.: US 2026/0041654 A1
(43) Pub. Date: Feb. 12, 2026

(54) METHOD FOR PREVENTING OR TREATING INFLAMMATORY SKIN DISEASE

(71) Applicant: GONGWIN BIOPHARM CO., LTD, Taipei City (TW)

(72) Inventors: Chuan-Ching YANG, Taipei City (TW); Shun-Chi WU, Taipei City (TW); Zong-Yu YANG, Taipei City (TW); Chia-En LIN, Taipei City (TW); Mao-Yuan LIN, Taipei City (TW)

(73) Assignee: GONGWIN BIOPHARM CO., LTD, Taipei City (TW)

(21) Appl. No.: 18/796,666

(22) Filed: Aug. 7, 2024

(51) Int. Cl. A61K 31/18; A61K 9/00; A61K 47/10; A61K 47/32; A61K 47/40; A61P 17/00; A61P 17/06; A61P 29/00

(52) U.S. Cl. CPC A61K 31/18; A61K 9/0014; A61K 47/10; A61K 47/32; A61K 47/40; A61P 17/00; A61P 17/06; A61P 29/00

(57) ABSTRACT

Provided is a method for preventing or treating an inflammatory skin disease, including administering to a subject in need thereof with an effective amount of a composition that includes a benzenesulfonamide derivative and a pharmaceutically or cosmetically acceptable excipient thereof. Also provided is a use of the composition in the manufacture of a medicament or a cosmetic for preventing or treating the inflammatory skin disease.

### BACKGROUND

### 1. Technical Field

The present disclosure relates to a method for preventing or treating a skin disease, and particularly to a method for preventing or treating an inflammatory skin disease.

### 2. Description of Associated Art

An inflammatory skin disease is the most common problem in dermatology, and the conditions of the inflammatory skin disease include from occasional rashes accompanied by itching and redness to chronic symptoms such as hives (urticaria), eczema (such as atopic dermatitis, neurodermatitis, nummular eczema, etc.) and psoriasis.

Methods for treating atopic dermatitis include use of humidifier, application of emollients, topical therapy such as topical corticosteroids for the first line therapy and topical calcineurin inhibitors for the second line therapy, phototherapy such as irradiation of ultraviolet A (UVA) and/or ultraviolet B (UVB) or extracorporeal photopheresis (ECP), etc. However, use of humidifier or application of emollients may not efficiently treat the topical dermatitis. In addition, the most common side effect of topical corticosteroids is a burring or stinging sensation when the topical corticosteroid is applied. Further, the equipment for UVA and/or UVB irradiation and ECP are not available to the patients; particularly, ECP is generally only available at tertiary care centers.

Therefore, developing a topical formulation which is easily available to the patients and provides anti-inflammation effects for inflammatory skin disease treatment without causing adverse reaction or unwanted side effects is an urgent problem to be solved in the art.

### SUMMARY

The present disclosure is based, at least in part, on the innovation that a composition comprising a benzenesulfonamide derivative has anti-inflammation effects.

The present disclosure provides a method for preventing or treating an inflammatory skin disease, comprises administering an effective amount of a composition to a subject in need thereof, wherein the composition comprises a benzenesulfonamide derivative and a pharmaceutically or cosmetically acceptable excipient thereof.

In at least one embodiment of the present disclosure, the benzenesulfonamide derivative in the topical formulation may be represented by following formula (I):

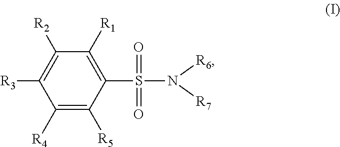

or a pharmaceutically acceptable salt thereof,
wherein R₁ to R₇ are independently selected from the group consisting of H, a C₁-C₆ linear or branched alkyl group, a C₁-C₆ linear or branched alkoxy group, a C₃-C₆ cycloalkyl group, a C₃-C₆ cycloheteroalkyl group, an amino group, and a halo group, or R₆ and R₇ are linked to each other to form a ring, and wherein the alkyl group, the alkoxy group, the cycloalkyl group, the cycloheteroalkyl group, and the ring in R₁ to R₇ are unsubstituted or substituted with one or more substituents.

In some embodiment of the present disclosure, the benzenesulfonamide derivative may be selected from the group consisting of para-toluene sulfonamide (p-TSA), ortho-toluene sulfonamide, meta-toluene sulfonamide, N-ethyl-ortho-toluene sulfonamide, N-ethyl-para-toluene sulfonamide, and N-cyclohexyl-para-toluene sulfonamide.

In some embodiments of the present disclosure, also provides a method for inhibiting inflammation on skin, the method comprises topically administering the composition to the skin of the subject in need thereof to inhibit inflammation on the skin. The present disclosure provides an anti-inflammatory regimen that renders an effective treatment, and provides less irritation compared with conventional therapies.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present disclosure can be more fully understood by reading the following detailed description of the embodiments, with reference made to the accompanying drawings, wherein:

FIG. 1 shows a graph of a cumulative amount of p-TSA in the skin of nude mice after applying the p-TSA-containing topical formulation having 1% of various surfactants to the skin of the nude mice;

FIG. 2A shows the layout of lipopolysaccharide (LPS)-induced tumor necrosis factor-α (TNF-α) quantitation assay;

FIGS. 2B and 2C show the production of LPS-induced TNF-α and the percentage of inhibition of LPS-induced TNF-α, respectively;

FIG. 3A shows the layout of cell viability assay;

FIG. 3B shows cell viability of RAW 264.7 macrophage cells after treatment with p-TSA in various concentrations;

FIGS. 4A and 4B show the inner wrist of a subject before (FIG. 4A) and after (FIG. 4B) the treatment of the p-TSA-containing topical formulation;

FIGS. 5A and 5B show the thumb back (dorsal skin) of a subject before (FIG. 5A) and after (FIG. 5B) the treatment of the p-TSA-containing topical formulation;

FIGS. 6A and 6B show the chin of another subject before (FIG. 6A) and after (FIG. 6B) the treatment of the p-TSA-containing topical formulation;

FIG. 7 shows the weight (g) change of Imiquimod-induced psoriasis model mice in various p-TSA treatment groups (Vasline, 5% IMQ, Vaseline+100% DMSO, 5% IMQ+100% DMSO, 5% IMQ+100% DMSO+4M p-TSA, vaseline+70% DMSO, 5% IMQ+70% DMSO, 5% IMQ+70% DMSO+1.65M p-TSA);

FIG. 8 shows the PASI score of Imiquimod-induced psoriasis model mice in various treatment groups (Vasline, 5% IMQ, Vaseline+100% DMSO, 5% IMQ+100% DMSO, 5% IMQ+100% DMSO+4M p-TSA, vaseline+70% DMSO, 5% IMQ+70% DMSO, 5% IMQ+70% DMSO+1.65M p-TSA);

FIG. 9A shows the change in epidermal thickness (mm) of Imiquimod-induced psoriasis model mice in various p-TSA treatment groups (Vasline, 5% IMQ, Vaseline+100% DMSO, 5% IMQ+100% DMSO, 5% IMQ+100% DMSO+4M p-TSA, vaseline+70% DMSO, 5% IMQ+70% DMSO, 5% IMQ+70% DMSO+1.65M p-TSA);

FIG. 9B shows the epidermal thickness (mm) of Imiquimod-induced psoriasis model mice in various p-TSA treatment group on the day of sacrifice;

FIGS. 10A-10C are images of H&E staining of skin of Imiquimod-induced psoriasis model mice treated with VC (Vaseline, animal ID: 1). FIG. 10A scale bar measures 200 μm; FIGS. 10B and 10C scale bars measure 50 μm;

FIGS. 11A-11C are images of H&E staining of skin of Imiquimod-induced psoriasis model mice treated with IC (5% IMQ, animal ID: 7). FIG. 11A scale bar measures 200 μm; FIGS. 11B and 11C scale bars measure 50 μm;

FIGS. 12A-12C are images of H&E staining of skin of Imiquimod-induced psoriasis model mice treated with HDC (Vaseline+100% DMSO, animal ID: 10). FIG. 12A scale bar measures 200 μm; FIGS. 12B and 12C scale bars measure 50 μm;

FIGS. 13A-13C are images of H&E staining of skin of Imiquimod-induced psoriasis model mice treated with IHD (5% IMQ+100% DMSO, animal ID: 13). FIG. 13A scale bar measures 200 μm; FIGS. 13B and 13C scale bars measure 50 μm;

FIGS. 14A-14C are images of H&E staining of skin of Imiquimod-induced psoriasis model mice treated with 14 MP (5% IMQ+100% DMSO+4 Mp-TSA, animal ID: 20). FIG. 14A scale bar measures 200 μm; FIGS. 14B and 14C scale bars measure 50 μm;

FIGS. 15A-15C are images of H&E staining of skin of Imiquimod-induced psoriasis model mice treated with LDC (Vaseline+70% DMSO, animal ID: 22). FIG. 15A scale bar measures 200 μm; FIGS. 15B and 15C scale bars measure 50 μm;

FIGS. 16A-16C are images of H&E staining of skin of Imiquimod-induced psoriasis model mice treated with ILD (5% IMQ+70% DMSO, animal ID: 25). FIG. 16A scale bar measures 200 μm; FIGS. 16B and 16C scale bars measure 50 μm;

FIGS. 17A-17C are images of H&E staining of skin of Imiquimod-induced psoriasis model mice treated with 11.6MP (5% IMQ+70% DMSO+1.6 Mp-TSA, animal ID: 32). FIG. 17A scale bar measures 200 μm; FIGS. 17B and 17C scale bars measure 50 μm;

FIG. 18 shows experimental time-course for DNCB-induced dermatitis model and topical application of p-TSA;

FIG. 19 are photographs of cutaneous manifestations (upper panel) and spleen (lower panel) of DNCB-induced dermatitis model in BALB/c mice in each group at Day 28 (DNCB only, DNCB+ vehicle treatment, and DNCB+1.6M p-TSA treatment);

FIG. 20 shows spleen weight of DNCB-induced dermatitis model in each treatment group;

FIG. 21 shows epidermal thickness (mm) of DNCB-induced dermatitis model in each treatment group;

FIG. 22 shows number of scratching in 10 minutes of DNCB-induced dermatitis model in each BALB/c mice group at Day 14 (before treatment);

FIG. 23 shows scratching behavior duration of DNCB-induced dermatitis model in each BALB/c mice group at Day 14 (before treatment);

FIG. 24 shows number of scratching in 10 minutes of DNCB-induced dermatitis model in each BALB/c mice group at Day 27 (after treatment);

FIG. 25 shows scratching behavior duration of DNCB-induced dermatitis model in each BALB/c mice group at Day 27 (after treatment);

### DETAILED DESCRIPTION OF THE EMBODIMENTS

The following examples are used to exemplify the present disclosure. A person of ordinary skill in the art can conceive the other advantages of the present disclosure, based on the disclosure in the specification of the present disclosure. The present disclosure can also be implemented or applied as described in different examples. It is possible to modify and/or alter the above examples for carrying out this disclosure without contravening its spirit and scope for different aspects and applications.

As used herein, the singular forms “a,” “an,” and “the” include plural referents unless expressly and unequivocally limited to one referent. The term “or” is used interchangeably with the term “and/or” unless the context clearly indicates otherwise.

As used herein, the terms “comprise,” “comprising,” “include,” “including,” “have,” “having,” “contain,” “containing,” and any other variations thereof are intended to cover a non-exclusive inclusion. For example, when describing an object “comprises” a limitation, unless otherwise specified, it may additionally include other ingredients, elements, components, structures, regions, parts, devices, systems, steps, or connections, etc., and should not exclude other limitations.

As used herein, the term “an effective amount” is the quantity of an active agent which achieves a clinical outcome when the compound is administered to a subject. For example, when an active agent of the disclosure is administered to a subject with a eczema, a favorable “clinical outcome” includes reduction in skin rashes and redness, reduction in itching, reduction in scaly skin patches, reduction in the severity of the symptoms associated with the eczema and/or increase in the well-being of the subject. The effective amount may vary, as recognized by those skilled in the art, depending on routes of administration, excipient usage, the possibility of co-usage with other therapeutic treatment, and the condition to be treated.

As used herein, the term “preventing” or “prevention” and the like refer to preventive or avoidance measures for a disease or symptoms or conditions of a disease, which include, but are not limited to, applying or administering one or more active agents to a subject who has not yet been diagnosed as a patient suffering from the disease or the symptoms or conditions of the disease but may be susceptible or prone to the disease. The preventive measures of the present disclosure are provided to avoid, prevent, or postpone the occurrence of the disease or the symptoms or conditions of the disease.

According to the disclosure, the terms “treatment,” “treating” and the like are used herein to generally mean obtaining a desired pharmacologic or physiologic effect. The effect may be prophylactic in terms of completely or partially preventing a condition, appearance, disease or symptom and/or may be therapeutic in terms of a partial or complete cure for a condition and/or adverse effect attributable to a condition or disease. The term “treatment” as used herein covers any treatment of a condition, disease or undesirable appearance in a mammal, e.g., a human, and includes: (a) inhibiting the disease, condition or symptom, i.e., causing regression of a condition or symptom; and (b) relieving the disease, condition or symptom, i.e., causing regression of a condition or symptom.

It is further noted that, the term “PTS” is used interchangeably with the term “p-TSA” or “pTSA” unless the context clearly indicates otherwise.

The numeral ranges used herein are inclusive and combinable, any numeral value that falls within the numeral scope herein could be taken as a maximum or minimum value to derive the sub-ranges therefrom. For example, it should be understood that the numeral range “1-60%” comprises any sub-ranges between the minimum value of 1% to the maximum value of 60%, such as the sub-ranges from 1% to 50%, from 10% to 60%, and from 20.5% to 40.5%.

The term “about” as used herein when referring to the numerical value is meant to encompass variations of ±20%, ±10%, ±5%, ±1%, ±0.5%, or ±0.1% from the numerical value. Such variations in the numerical value may occur by, e.g., the experimental error, the typical error in measuring or handling procedure for making compounds, compositions, concentrates, or formulations, the differences in the source, manufacture, or purity of starting materials or ingredients used in the present disclosure, or like considerations.

As used herein, “subject” may encompass any vertebrate including, but not limited to, mammals, reptiles, amphibians, and/or fish. However, advantageously, the subject is a mammal such as a human, or an animal mammal such as a domesticated mammal, e.g., a dog, a cat, a horse, or the like, or a production mammal, e.g., a cow, a sheep, a pig, or the like.

The present disclosure provides a method for preventing or treating an inflammatory skin disease, comprising administering an effective amount of a composition to a subject in need thereof, wherein the composition comprises a benzenesulfonamide derivative and a pharmaceutically or cosmetically acceptable excipient thereof.

As used herein, the term “an inflammatory skin disease” refers to a disease causing the symptoms from occasional rashes accompanied by itching and redness to chronic symptoms. In at least one embodiment of the present disclosure, the inflammatory skin diseases include herpes simplex, shingles, eczema such as atopic dermatitis, psoriasis, urticarial, atopic dermatitisor any combination thereof, but the present disclosure is not limited thereto. In some embodiments, the inflammatory skin disease is eczema such as atopic dermatitis, psoriasis, atopic dermatitis, or any combination thereof. In some embodiments, the inflammatory skin disease exclude acne and/or other bacterial-induced inflammatory skin disease. In some embodiment of the present disclosure, the symptoms of the inflammatory skin disease may occur in any body surface of the subject, for example wrist, fingers or face.

In at least one embodiment of the present disclosure, the benzenesulfonamide derivative is represented by formula (I):

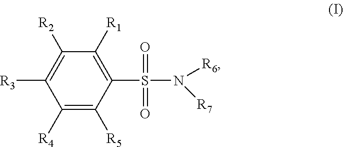

or a pharmaceutically acceptable salt thereof,
wherein R₁ to R₇ are independently selected from the group consisting of H, a C₁-C₆ linear or branched alkyl group, a C₁-C₆ linear or branched alkoxy group, a C₃-C₆ cycloalkyl group, a C₃-C₆ cycloheteroalkyl group, an amino group, and a halo group, or R₆ and R₇ are linked to each other to form a ring.

In at least one embodiments of the present disclosure, the alkyl group, the alkoxy group, the cycloalkyl group, the cycloheteroalkyl group, and the ring in R₁ to R₇ are independently unsubstituted or substituted with one or more substituents. In some embodiments, the substituent is selected from the group consisting of phenyl, halo, oxo, ether, hydroxyl, carboxyl, amino, sulfo, and sulfonamide groups.

In at least one embodiment of the present disclosure, the benzenesulfonamide derivative may be, but not limited to, para-toluenesulfonamide, ortho-toluenesulfonamide, meta-toluenesulfonamide, N-ethyl-para-toluene sulfonamide, N-ethyl-ortho-toluene sulfonamide, or N-cyclohexyl-para-toluene sulfonamide.

In at least one embodiment of the present disclosure, the composition agent is administered to the subject topically, orally, intravenously, subcutaneously, intradermally, orally, intrathecally, intraperitoneally, intranasally, intramuscularly, intrapleuraly, topically, or through nebulization. In some embodiments, the composition agent is administered to the subject topically.

In at least one embodiment of the present disclosure, the pharmaceutically or cosmetically acceptable excipient may be a preservative, a lubricant, a suspending agent, a wetting agent, a flavoring agent, a thickening agent, a biocompatible solvent, a surfactant, a complexation agent, or any combination thereof.

In at least one embodiment of the present disclosure, the preservatives may include, but are not limited to, sodium benzoate, methyl paraben, propyl paraben, and cresols.

In at least one embodiment of the present disclosure, the lubricant may be metallic stearates which include, but are not limited to, magnesium, calcium and sodium stearates, stearic acid, talc, polyethylene glycols, and soluble salts. In another embodiment of the present disclosure, the salts include sodium chloride or sodium benzoate.

In at least one embodiment of the present disclosure, the wetting agents may include, but are not limited to, glycerol, sorbitol, and polypropylene glycol.

In at least one embodiment of the present disclosure, the flavoring agents may include, but are not limited to, peppermint oil, menthol, lemon oil, orange oil, and cinnamon oil.

In at least one embodiment of the present disclosure, the thickening agents may include, but are not limited to, SEPINEO P600, SEPINEO DERM, CARBOPOL 980, sodium carboxymethyl cellulose (Na CMC), xanthan gum, hydroxypropyl cellulose (HPC), and polyvinylpyrrolidone K90 (PVP K90).

As used herein, the term “biocompatible” means generation of no significant undesirable host response for the intended utility. For example, biocompatible materials are non-toxic for the intended utility. Thus, for human utility, biocompatible is more preferably non-toxic to humans or human tissues.

In at least one embodiment of the present disclosure, the biocompatible solvent may be polyethylene glycol, propylene glycol, glycerol, sorbitol, ethanol, dimethyl sulfoxide, N-methyl-2-pyrrolidone (NMP), N,N-dimethylacetamide (DMA), glycofurol, acetone, isopropyl alcohol (IPA), triglyceride, benzyl benzoate, benzyl alcohol, solketal or any combination thereof.

In at least one embodiment of the present disclosure, the surfactant may be lecithin, macrogol 15 hydroxystearate, polyoxyethylene alkyl ether, polyoxyethylene castor oil, polyoxyethylene sorbitan fatty acid ester, polyoxyethylene stearate, polyoxylglyceride, sorbitan ester, tocopheryl polyethylene glycol succinate (TPGS) or any combination thereof.

In one embodiment of the present disclosure, the complexation agent may be polyvinyl pyrrolidone or cyclodextrin.

In at least one embodiment of the present disclosure, the benzenesulfonamide derivative is present in an amount ranging from about 1% to about 70% by weight. In some embodiments of the present disclosure, the benzenesulfonamide derivative is present in an amount ranging from about 1% to about 60% by weight. In some embodiments of the present disclosure, the benzenesulfonamide derivative is present in an amount ranging from about 10% to about 60% by weight. In another embodiment of the present disclosure, the benzenesulfonamide derivative is present in an amount of 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, or 70% by weight.

In at least one embodiment of the present disclosure, the pharmaceutically or cosmetically acceptable excipient is present in an amount ranging from about 30% to about 99% by weight. In some embodiments of the present disclosure, the pharmaceutically or cosmetically acceptable excipient is present in an amount ranging from about 40% to about 90% by weight.

In another embodiment of the present disclosure, the pharmaceutically or cosmetically acceptable excipient is present in an amount ranging from about 50% to about 85% by weight. In still another embodiment of the present disclosure, the pharmaceutically or cosmetically acceptable excipient is present in an amount ranging from about 60% to about 75% by weight.

In at least one embodiment of the present disclosure, the topical formulation is in a form suitable for topical administration. In another embodiment of the present disclosure, the topical formulation may be in a form of a gel, a soft gel, a lotion, an ointment, a paste, an emulsion, a cream, a slurry, a patch, a film, a cream, or an aerosol.

In some embodiments of the present disclosure, the topical formulation is used as a pharmaceutical product or a cosmetic.

In at least one embodiment of the present disclosure, the composition is administered to the subject 7 times to 42 times a week or 1 time to 6 times a day. In some embodiments, the composition containing the benzenesulfonamide derivative is topically applied to an inflamed area of the subject's skin once or twice daily or as needed to treat the inflammatory skin disease of the subject. In some embodiments, the composition is continuously administered to the subject for at least 1 week, 2 weeks or 4 weeks.

The present disclosure also provides a use of a composition in the manufacture of a medicament or a cosmetic for preventing or treating an inflammatory skin disease, and the composition comprises the benzenesulfonamide derivative and the pharmaceutically acceptable excipient thereof as mentioned above. The present disclosure further provides the pharmaceutical composition for use in the prevention or treatment of the inflammatory skin disease, and the pharmaceutical composition comprises the benzenesulfonamide derivative and the pharmaceutically or cosmetically acceptable excipient thereof as mentioned above.

The following are embodiments further demonstrating the efficacy of the current disclosure, but not to limit the scope of the present disclosure.

### EXAMPLE

The present disclosure has been described using exemplary embodiments. However, it is to be understood that the scope of the disclosure is not limited to the disclosed embodiments. On the contrary, it is intended to cover various modifications and similar rearrangements. The scope of the claims therefore should be accorded the broadest interpretation so as to encompass all such modifications and similar rearrangements.

### Example 1: Measuring Solubility of p-Toluenesulfonamide (p-TSA) in Various Solvents

To screen the biocompatible solvents useful for preparation of the p-TSA-containing topical formulation, p-TSA was dissolved in various solvents at a maximum concentration. The results showed that dimethyl sulfoxide, polyethylene glycol 400 (PEG-400), glycofurol 75, propylene glycol, glycerol, absolute ethanol and 20% 7-sulfobutyl-ether-β-cyclodextrin were biocompatible solvents suitable for dissolving p-TSA. The solubility of p-TSA in such biocompatible solvents was shown in Table 1.

TABLE 1
Solubility of p-TSA in various solvents (n = 3)

| Solvents | Solubility (mg/mL) |
|---|---|
| Dimethyl sulfoxide (DMSO) | 785.95 |
| Polyethylene glycol 400 (PEG-400) | 466.52 |
| Glycofurol 75 | 443.13 |
| Propylene glycol (PG) | 117.86 |
| Glycerol | 28.42 |
| Absolute ethanol | 107.61 |
| 20% 7-sulfobutyl-ether-β-cyclodextrin | 17.59 |
| (SBEβCD) | |

### Example 2: Development of p-TSA-Containing Topical Formulation

To develop a p-TSA-containing topical formulation in a gel form, 15%, 25% and 40% p-TSA were individually dissolved in different combination of PEG-400, DMSO and water with 3% SEPINEO P600 as a thickening agent. The dissolution and precipitation behaviors of different batches of p-TSA-containing topical formulations were shown in Table 2. The results showed that batches 15-2 and 25-2 of the topical formulations cannot completely dissolve the corresponding 15% and 25% p-TSA contained in their formulations, while significant precipitation was found in batch 15-1 of the topical formulation.

TABLE 2
Dissolution profiles of p-TSA in various solvents (n = 3)

| | Batch | | | | |
|---|---|---|---|---|---|
| Components | 15-1 | 15-2 | 25-1 | 25-2 | 40 |
| p-TSA | 15% | 15% | 25% | 25% | 40% |
| PEG-400 | 50% | 20% | 50% | 40% | 30% |
| DMSO | 0% | 0% | 10% | 0% | 20% |
| SEPINEO P600 | 3% | 3% | 3% | 3% | 3% |
| H₂O | 32% | 62% | 12% | 32% | 7% |
| Dissolution | Dissolved | Incompletely | Dissolved | Incompletely | Dissolved |
| behavior | | dissolved | | dissolved | |
| Precipitation* | N.D. | N.D. | N.D. | N.D. | N.D. |

1. *Drug precipitation within the topical formulation was examined by use of a light microscope.
2. N.D. is defined as not detected.
3. % means the percentage by weight.

To further examine the characterization of p-TSA gel, 25% p-TSA dissolved in 62% PEG-400 with various thickening agents, including SEPINEO P600, SEPINEO DERM, CARBOPOL 980, sodium carboxymethyl cellulose (Na CMC), xanthan gum, hydroxypropyl cellulose (HPC) and polyvinylpyrrolidone K90 (PVP K90) were prepared. The sample appearance and precipitation behavior of the p-TSA gels were observed, and the results were shown in Table 3. The results showed that stable topical formulation in gel form was successfully manufactured by dissolving 25% p-TSA in 62% PEG-400 with 3% SEPINEO P600 or 3% SEPINEO DERM (batches 25-5 and 25-6).

TABLE 3
Characterization of p-TSA-containing topical formulations having various thickening agents

| | Batch | | | | | | |
|---|---|---|---|---|---|---|---|
| Components | 25-5 | 25-6 | 25-7 | 25-8 | 25-9 | 25-10 | 25-11 |
| p-TSA | 25% | 25% | 25% | 25% | 25% | 25% | 25% |
| PEG-400 | 62% | 62% | 62% | 62% | 62% | 62% | 62% |
| Thickening | 3% | 3% | 3% | 3% | 3% | 3% | 3% |
| agent | SEPINEO | SEPINEO | CARBOPOL | Na CMC | Xanthan | HPC | PVP K90 |
| | P600 | DERM | 980 | | gum | | |
| H₂O | 10% | 10% | 10% | 10% | 10% | 10% | 10% |
| Appearance | Translucent | Translucent | Translucent | Opaque | Opaque | Highly | Transparent, |
| | gel | gel | gel with few | suspension, | suspension, | viscous | viscous |
| | | | white powder | sediment | sediment | solution, in | solution |
| | | | clumps | occurred | occurred | the | |
| | | | | after | after | presence of | |
| | | | | sample | sample | transparent | |
| | | | | standing | standing | gel-like | |
| | | | | for a while | for a while | stuffs | |
| Precipitation* | Nil | Nil | Nil | Substantial | Substantial | Nil | Nil |

1. *Drug precipitation within the topical formulation was examined by use of a light microscope.
2. % means the percentage by weight.

### Example 3: Penetrability of p-TSA-Containing Topical Formulation

To examine the penetration effect of a p-TSA-containing topical formulation in the gel form, 25% p-TSA was dissolved in 62% PEG-400 with 1% of various surfactants, including Transcutol HP, lecithin and Kolliphor RH40 (BASF Corporation) as shown in Table 4.

TABLE 4
Components of p-TSA-containing topical
formulations having various surfactants

| Components | Sample 1 | Sample 2 | Sample 3 | Sample 4 |
|---|---|---|---|---|
| p-TSA | 25% | 25% | 25% | 25% |
| PEG-400 | 62% | 62% | 62% | 62% |
| Surfactant | — | 1% | 1% | 1% |
| | | Transcutol HP | Lecithin | Kolliphor RH40 |
| H₂O | 13% | 12% | 12% | 12% |

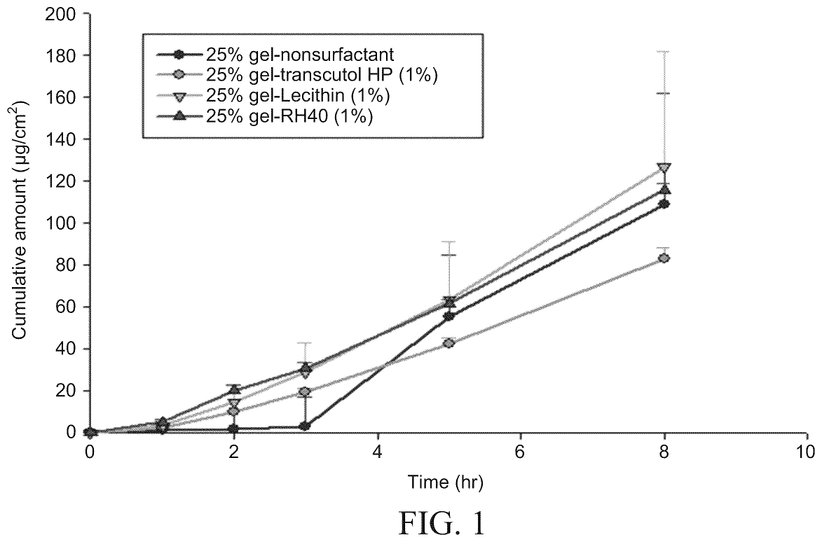

Further, 10 mg of each of Samples 1˜4 was respectively applied to 1 cm² area of the skin of nude mice, followed by measurement of the cumulative amount of p-TSA by HPLC. Referring to FIG. 1, the results showed that the surfactants, especially lecithin, rendered the penetrability to p-TSA topical formulation.

### Example 4: Anti-Inflammatory Activity of p-TSA-Containing Topical Formulation

p-TSA in different concentrations was used to test the anti-inflammatory effects on tumor necrosis factor-α (TNF-α) secretion in lipopolysaccharide (LPS)-stimulated mouse RAW 264.7 macrophage cells, and cell viability assay was conducted to verify the cytotoxicity effect of p-TSA. Briefly, the procedures of LPS-induced TNF-α quantitation and cell viability assay in this example were described as follows.

### 1. LPS-Induced TNF-α Quantitation (PerkinElmer, TRF1505C)

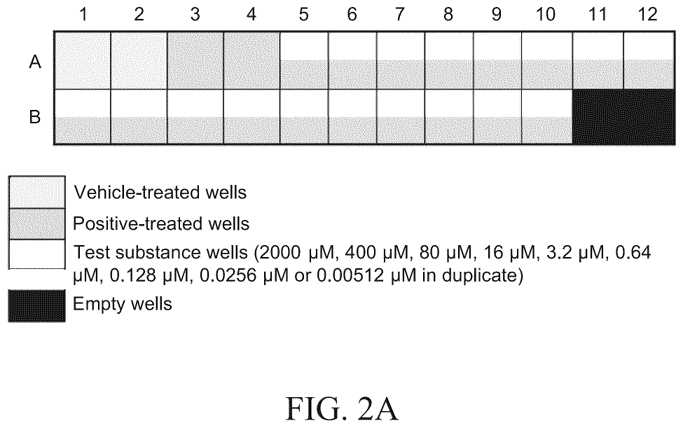

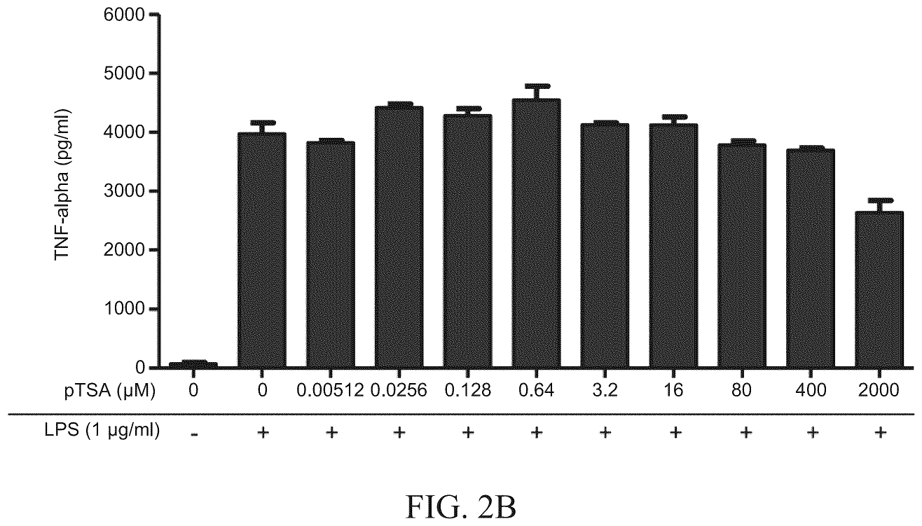

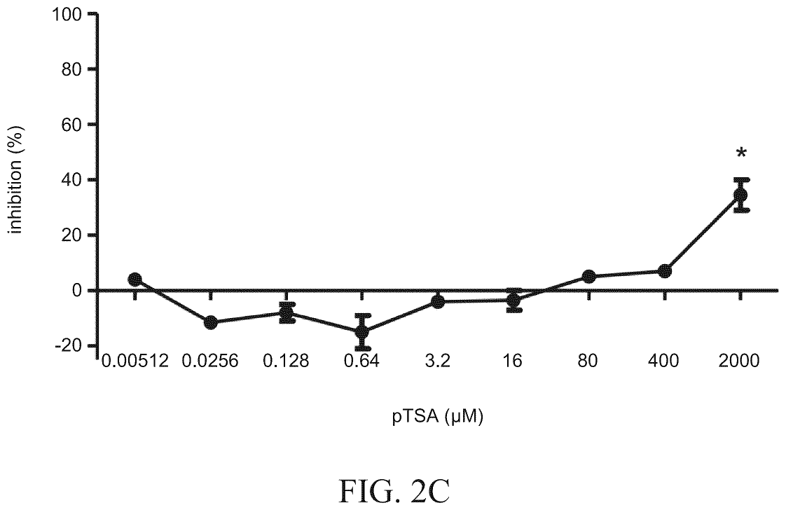

Test substance stock of p-TSA was prepared as 342 mg/ml (2 M) in 100% DMSO, and serial dilution was performed to obtain 9 concentration doses of the test substance with 2000 μM, 400 μM, 80 μM, 16 μM, 3.2 μM, 0.64 μM, 0.128 μM, 0.0256 μM and 0.00512 μM. Each of the test substance or vehicle (0.1% DMSO) was pre-incubated with 5×10³ RAW 264.7 macrophage cells (ATCC, TIB-71) in each well of a 96-well plate for 20 minutes, followed by addition of 1 μg/ml LPS (from Escherichia coli 055:B5, Sigma, L-2880) for initiating the reaction (referring to the layout shown in FIG. 2A). After 24 hours incubation, cell supernatants were harvested for TNF-α quantitation with appropriate dilution pursuant to the kit instructions. Test substance induced inhibition of LPS-induced TNF-α secretion was normalized to vehicle (100% inhibition) and 1 μg/ml LPS (0% inhibition), respectively. The results were shown in FIGS. 2B and 2C.

In FIG. 2B, a complete range of p-TSA from 2000 to 0 μM was applied to RAW264.7 cells (5×10³/well) and followed 1 μg/ml LPS was added to incubate for 24 hours. Cells produced TNF-alpha was quantitated following kit's instructions. The result showed LPS induced TNF-alpha production in RAW264.7 cells in the presence of titrated p-TSA. FIG. 2C. Inhibition of LPS induced TNF-alpha production by p-TSA

In FIG. 2C, a complete range of p-TSA from 2000 to 0 μM was applied to RAW264.7 cells (5×10³/well) with 1 μg/ml LPS. An approximately 30% inhibition effect on LPS-induced TNF-alpha production at the concentration of 2000 μM of pTSA was observed. *p<0.05 is calculated with respect to LPS-stimulated controls using student's t test.

### 2. Cell Viability Assay

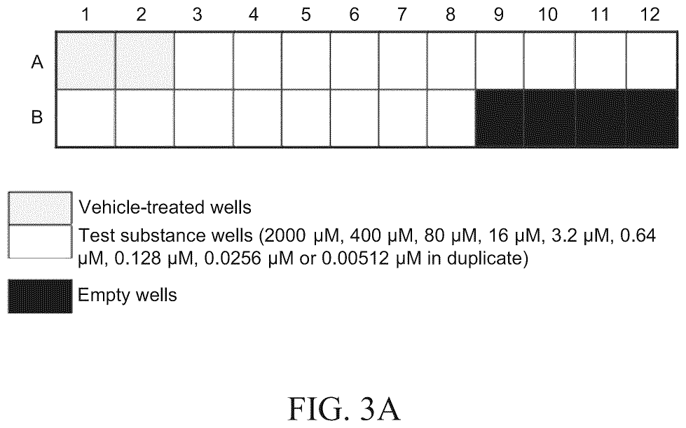

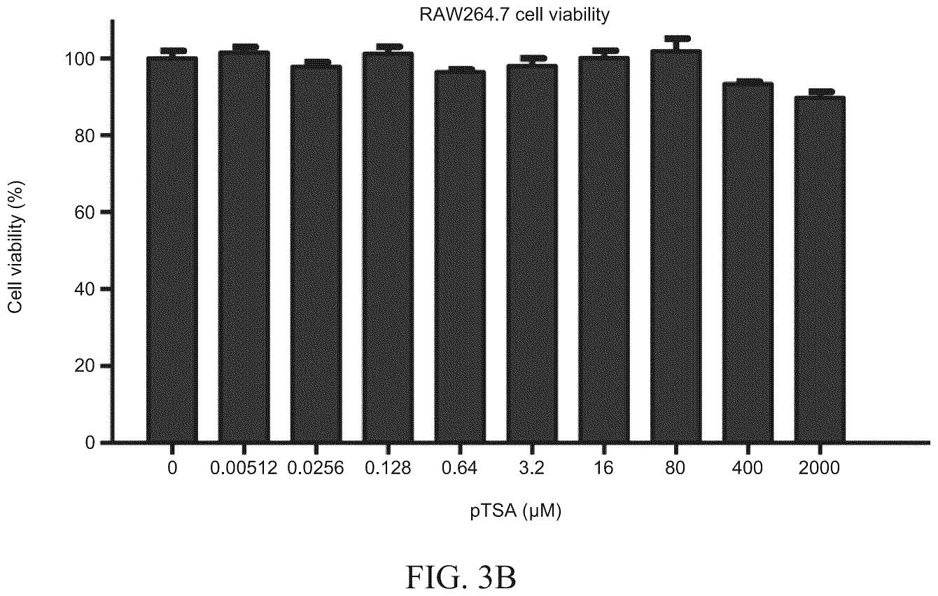

5×10³ RAW 264.7 macrophage cells were treated with test substance or vehicle (0.1% DMSO) in each well of a 96-well plate for 24 hours (referring to the layout shown in FIG. 3A). After incubation, the cell viability was measured by adding CellTiter-glo, and the luminescent signal was normalized to vehicle-treated cells (100% viability). Cell viability when cells are treated with dose-titrated pTSA was shown in FIG. 3B, specifically where a complete range of pTSA from 2000 to 0 μM was applied to RAW264.7 cells (5×10³/well). No significant cytoeffect on viability was observed.

### Example 5: Treatment of an Inflammatory Skin Disease by Use of p-TSA-Containing Topical Formulation

### Clinical Case I:

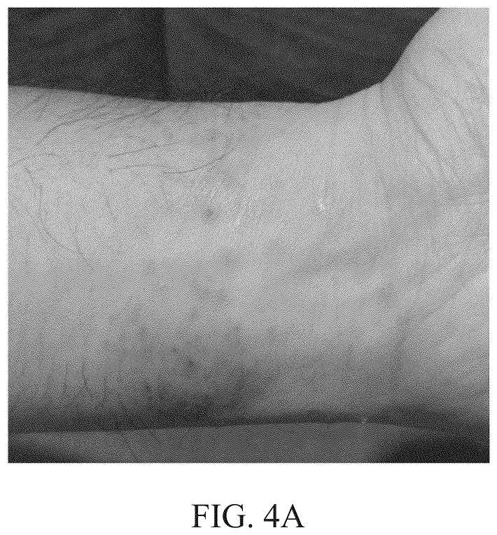

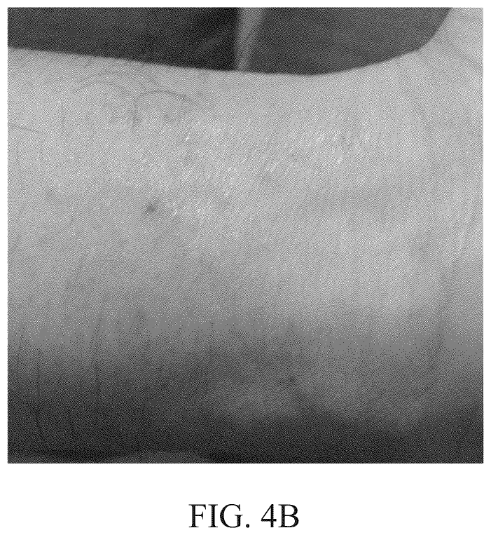

As shown in FIG. 4A, a subject was suffering from atopic dermatitis on the subject's inner wrist, and FIG. 4B showed that the severity of atopic dermatitis had been obviously reduced after applying an appropriate amount of 25% p-TSA gel (the batch 25-1 formulation of Table 2) evenly to the subject's inner wrist for 3 times a day within one week. In addition, the p-TSA gel did not cause skin irritation.

### Clinical Case II:

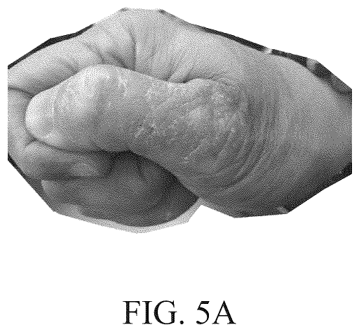

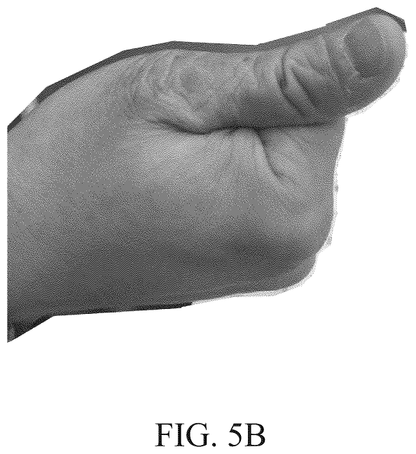

As shown in FIG. 5A, a subject was suffering from atopic dermatitis on the subject's thumb back, and FIG. 5B showed that the severity of atopic dermatitis had been obviously reduced after applying an appropriate amount of 40% p-TSA gel (the batch 40 formulation of Table 2) evenly to the subject's thumb back for 3 times a day within one week. In addition, the p-TSA gel did not cause skin irritation.

### Clinical Case III:

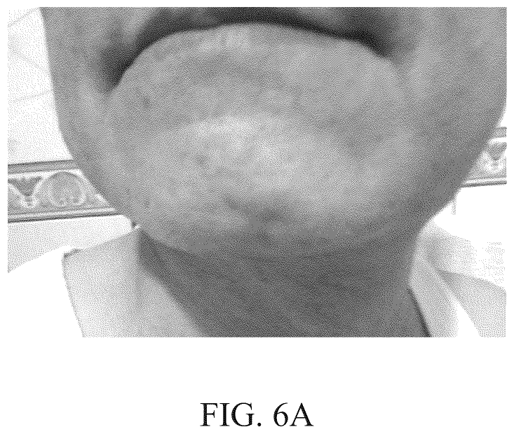

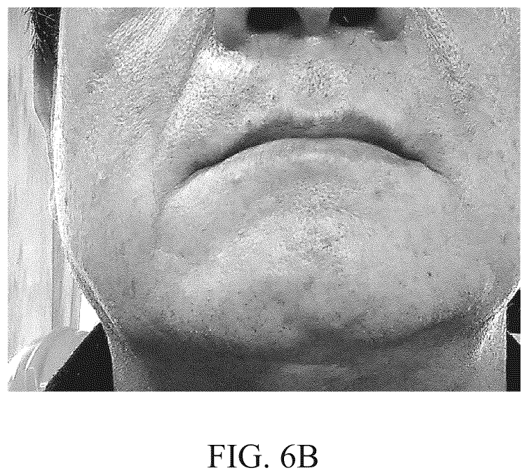

As shown in FIG. 6A, a subject was suffering from eczema on the subject's chin, and FIG. 6B showed that the severity of eczema had been obviously reduced after applying an appropriate amount of 25% p-TSA gel (the batch 25-1 formulation of Table 2) evenly to the subject's chin for 3 times a day within one week. In addition, the p-TSA gel did not cause skin irritation.

From the above clinical cases I to III, it was clearly showed that the p-TSA-containing topical formulation efficiently treated inflammatory skin diseases including eczema and atopic dermatitis in a subject without causing skin irritation.

### Example 6: Use of an Animal Model to Assess Efficacy of p-TSA in Treating Psoriasis

Imiquimod-induced psoriasis-like skin inflammation experiment with histology analysis.

### Material and Methods

### 1. Animals, Induction of Imiquimod-Induced Psoriasis, and Treatment

a. Mouse Entry:

Thirty-four female BALB/c mice at eight-weeks of age were obtained from BioLASCO, Taipei, Taiwan. Five mice each are housed in a cage with controlled environment and allowed to acclimatize before experimental use. After 1 week of acclimation period, body weight of the mice was measured the day before the experiment and the dorsal area of mice was shaved and depilated.

b. Induction Method

Mice are then placed in an anesthetic chamber and anesthetized using a mixture of air and 2% isoflurane at a rate of 1 Liter per minute. Once unconscious, the mice are transferred to a platform, where anesthesia is maintained through a mask. The hair on the dorsal area of the mice is shaved and a thin layer of depilatory cream are applied to the shaved area for 2 minutes. The cream and remaining of hair are wiped off with damped tissue paper. This is day 0 of the experiment. Then, on day 1 of the experiment, topical dose of 42 mg commercially available Imiquimod (IMQ) cream (5%) (Aldara; 3M Pharmaceuticals) are applied on the back daily for 7 consecutive days. See Table 5 for active ingredients used for the experiment.

TABLE 5
Active ingredient used for induction
of dermal irritation and treatment

| Active ingredient | Use | Cas Number | Manufacture |
|---|---|---|---|
| Para- | Experimental group | 70-55-3 | Sigma Aldrich |
| toluenesulfonamide | | | |
| 5% Imiquimod | Induce psoriasis | 99011-02-6 | 3M |
| cream | | | |

c. Mode of Administration

Start the administration on day 0 by using a cotton swab soaked with the liquid of the concentration to be tested, and applying it topically to psoriasis-induced areas according to Table 6.

TABLE 6
Treatment groups for Evaluation of p-TSA in Treating Psoriasis

| | Topical | | | | Dosing | Group |
|---|---|---|---|---|---|---|
| Group | application | Treatment | Concentration | N | frequency | abbreviation |
| 1 | Vaseline | — | | 3 | Daily | VC |
| 2 | 5% IMQ | | | 5 | Daily | IC |
| 3 | Vaseline | 100% DMSO | | 3 | Daily | HDC |
| 4 | 5% IMQ | 100% DMSO | | 5 | Daily | IHD |
| 5 | 5% IMQ | p-TSA | 4000 mM | 5 | Daily | I4MP |
| | | in 100% DMSO | | | | |
| 6 | Vaseline | 70% DMSO | | 3 | Daily | LDC |
| 7 | 5% IMQ | 70% DMSO | | 5 | Daily | ILD |
| 8 | 5% IMQ | p-TSA | 1650 mM | 5 | Daily | I1.6MP |
| | | in 70% DMSO | | | | |

d. Data Collection

Body weight: measure and record the body weight every day.

Epidermal thickness: the dorsal skin of the mice is divided into four quadrants and measured using a caliper

PASI scoring system: evaluate the severity of skin lesions in terms of erythema, scales and infiltration. To score the severity of inflammation of the back skin, an objective scoring system was developed based on the clinical Psoriasis Area and Severity Index (PASI). Erythema, desquamation (scaling), and induration (thickening) were scored independently on a scale from 0 to 4:0, normal skin; 1, Very mild erythema with very mild thickening and scaling involving a small area; 2, Mild erythema with mild thickening and scaling involving a small area; 3, Moderate erythema with moderate thickening and scaling (irregular and patchy) on whole back skin or skin lesions significantly overtop the normal skin; 4, Severe erythema with marked thickening and dense desquamation/severe scaling (irregular and patchy).

e. Data Analysis

Photographs are taken every day to record the disease severity with the PASI index of the mice.

Psoriasis-induced areas by administration of IMQ for 7 consecutive days starting from the day after shaving and sacrificed on the 8th day. The excised dorsal cutaneous tissue is fixed with 10% formalin for 48 hours and samples is then stained and submitted for histopathological examination by a veterinarian, see Tables 7-9 for grouping details and histopathological result.

Determination of therapeutic effect is based on the calculated difference in the epidermal thickness of the psoriatic skin between groups of mice on the day of sacrifice.

TABLE 7
Formalin-fixed skin samples of mice were submitted
for histopathological examination

| No | Group | Pathological number |
|---|---|---|
| 1 | VC | VC1 |
| 2 | | VC2 |
| 3 | | VC3 |
| 4 | IC | IC4 |
| 5 | | IC5 |
| 6 | | IC6 |
| 7 | | IC7 |
| 8 | | IC8 |
| 9 | HDC | HDC9 |
| 10 | | HDC10 |
| 11 | | HDC11 |
| 12 | IHD | IHD12 |
| 13 | | IHD13 |
| 14 | | IHD14 |
| 15 | | IHD15 |
| 16 | | IHD16 |
| 17 | I4MP | I4MP17 |
| 18 | I4MP | I4MP18 |
| 19 | | I4MP19 |
| 20 | | I4MP20 |
| 21 | | I4MP21 |
| 22 | LDC | LDC22 |
| 23 | | LDC23 |
| 24 | | LDC24 |
| 25 | ILD | ILD25 |
| 26 | | ILD26 |
| 27 | | ILD27 |
| 28 | | ILD28 |
| 29 | | ILD29 |
| 30 | I1.6MP | I1.6MP30 |
| 31 | | I1.6MP31 |
| 32 | | I1.6MP32 |
| 33 | | I1.6MP33 |
| 34 | | I1.6MP34 |

TABLE 8
Pathological nomenclatures
Observation fate

Gross finding:
No abnormalities (NA)
Left (L); Right (R)
Bilateral (B)
Slight, +
Mild, ++
Moderate, +++
Severe, ++++
Histopathological nomenclatures:
No significant lesions (NSL)
Modification: Degeneration, Necrosis, . . .
Distribution: Focal, Multifocal, Local Extensive and Diffuse
Degree: Minimal, Slight, Moderate, Moderate/Sever and Severe/High
Duration: Acute, Subacute, and Chronic
Exudate: Serous, Fibrinous, and Purulent

Severity of lesions was graded according to the methods described by Shackelford et al. (2002) (Toxicologic Pathology 30:93-96, 2002). Skin samples were cut into 4 μm sections and stained with hematoxylin-eosin (H&E) reagents. Degree of lesions stained with H&E was graded from one to five depending on severity: 1=minimal (<1%); 2: slight (1-25%); 3=moderate (26-50%); 4=moderate/severe (51-75%); 5=severe/high (76-100%).

TABLE 9
Pathology: individual micro findings of skin samples were scored by H&E staining

| | | Pathological number | | | | | | | | | | | | | | | |
|---|---|---|---|---|---|---|---|---|---|---|---|---|---|---|---|---|---|
| | Histopathological | VC | | | IC | | | | | HDC | | | IHD | | | | |
| Organ | finding | 1 | 2 | 3 | 4 | 5 | 6 | 7 | 8 | 9 | 10 | 11 | 12 | 13 | 14 | 15 | 16 |
| Skin | Inflammation, | — | 1 | 1 | 4 | 4 | 4 | 4 | 4 | 3 | 2 | 2 | 4 | 4 | 4 | 4 | 4 |
| | neutrophil and | | | | | | | | | | | | | | | | |
| | mononuclear | | | | | | | | | | | | | | | | |
| | cell, dermis | | | | | | | | | | | | | | | | |
| | and subcutis | | | | | | | | | | | | | | | | |
| | Hyerplasia, | — | — | — | 3 | 3 | 3 | 3 | 3 | 2 | 2 | 1 | 3 | 3 | 3 | 3 | 3 |
| | epidermis, and | | | | | | | | | | | | | | | | |
| | follicular | | | | | | | | | | | | | | | | |
| | epithelium | | | | | | | | | | | | | | | | |
| | Hyperkeratosis, | — | — | — | 3 | 2 | 2 | 3 | 2 | 1 | 1 | 1 | 2 | 3 | 3 | 2 | 2 |
| | epidermis | | | | | | | | | | | | | | | | |

| | | Pathological number | | | | | | | | | | | | | | | | | |
|---|---|---|---|---|---|---|---|---|---|---|---|---|---|---|---|---|---|---|---|
| | Histopathological | I4MP | | | | | LDC | | | ILD | | | | | I1.6MP | | | | |
| Organ | finding | 17 | 18 | 19 | 20 | 21 | 22 | 23 | 24 | 25 | 26 | 27 | 28 | 29 | 30 | 31 | 32 | 33 | 34 |
| Skin | Inflammation, | 3 | 3 | 4 | 3 | 3 | 2 | 1 | 3 | 4 | 4 | 4 | 4 | 4 | 4 | 4 | 4 | 4 | 3 |
| | neutrophil and | | | | | | | | | | | | | | | | | | |
| | mononuclear | | | | | | | | | | | | | | | | | | |
| | cell, dermis | | | | | | | | | | | | | | | | | | |
| | and subcutis | | | | | | | | | | | | | | | | | | |
| | Hyerplasia, | 3 | 2 | 3 | 2 | 2 | 1 | — | 1 | 3 | 3 | 3 | 3 | 3 | 3 | 3 | 3 | 3 | 2 |
| | epidermis, and | | | | | | | | | | | | | | | | | | |
| | follicular | | | | | | | | | | | | | | | | | | |
| | epithelium | | | | | | | | | | | | | | | | | | |
| | Hyperkeratosis, | 2 | 2 | 2 | 2 | 2 | 1 | — | 1 | 2 | 2 | 3 | 2 | 2 | 3 | 3 | 3 | 2 | 2 |
| | epidermis | | | | | | | | | | | | | | | | | | |

“—” No significant lesions. Degree of lesions stained with H&E was graded from one to five depending on severity: 1 = minimal (<1%); 2: slight (1-25%); 3 = moderate (26-50%); 4 = moderate/severe (51-75%); 5 = severe/high (76-100%).

### Result: P-TSA can Mitigate Imiquimod-Induced Psoriasis-Like Skin Inflammation

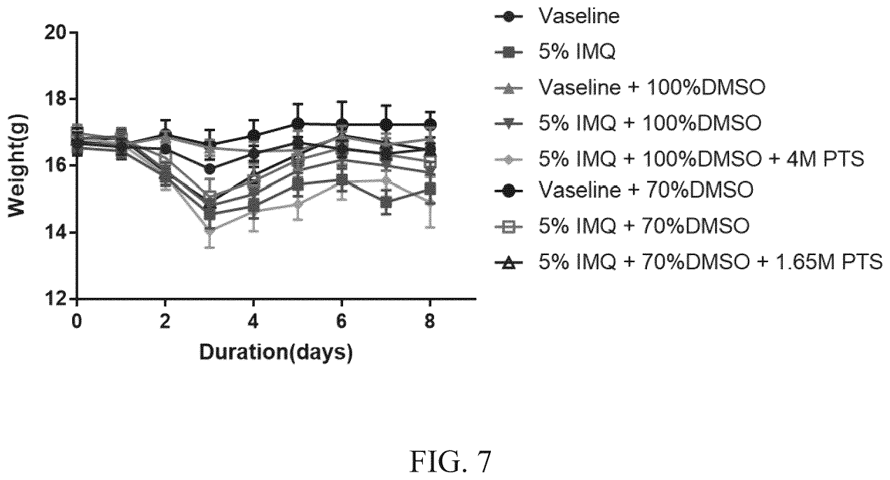

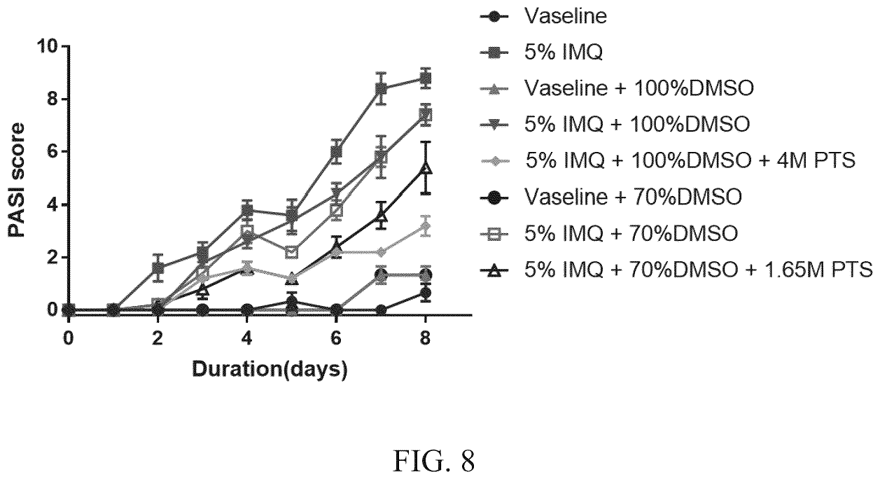

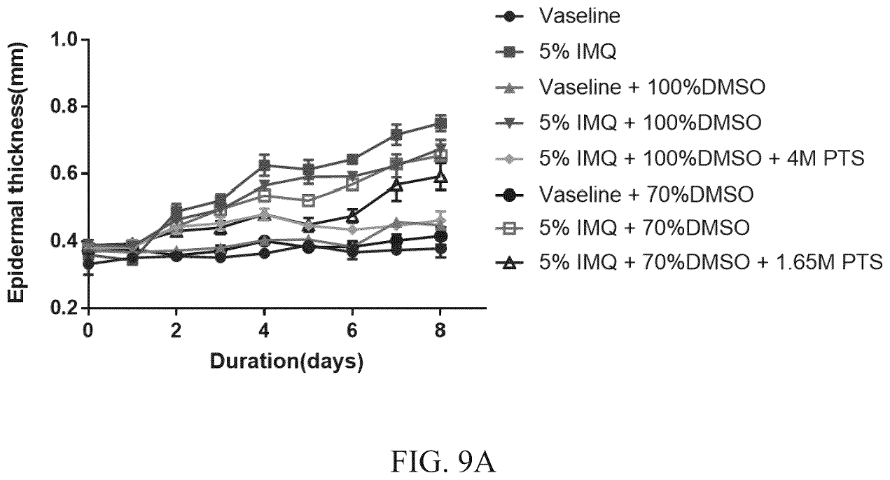

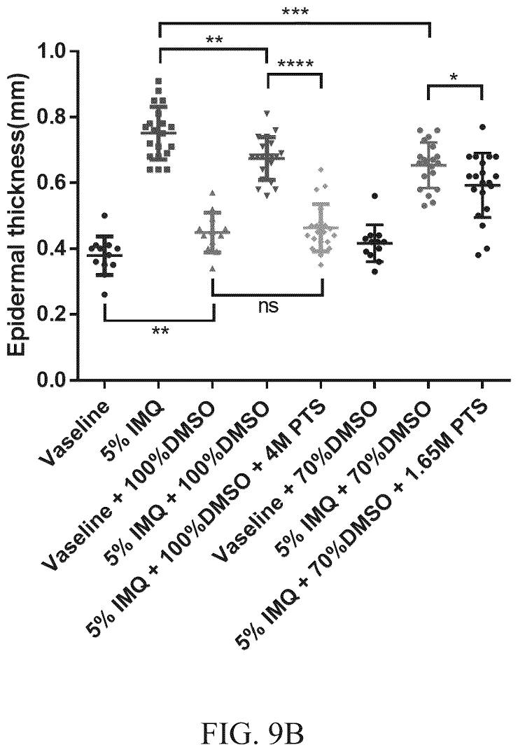

As observed for BALB/c mice, daily application of IMQ on the back on mice resulted in erythema, scaling, increased skin thickness and body weight loss. Specifically, as shown in FIG. 7, in all groups that had been treated with 5% IMQ, there were an observable drop in body weight as compared to groups that had not been treated with IMQ during the first few days, but there is no significant difference in weight change within the groups that had been treated with IMQ. The PASI scores of 4M and 1.65M p-TSA treatment groups (FIG. 8), and the change in epidermal thickness (FIGS. 9A and 9B) were reduced significantly, and the change of the PASI score and epidermal thickness are dose dependent. In other words, taken together with the result of Table 9, mice treated with 4M or 1.65M p-TSA significantly alleviated skin inflammation, as indicated in the reduction of PASI score as well as the reduction of classical psoriatic characteristics in pathological sections, including a reduction in skin lesions and epidermal thickness.

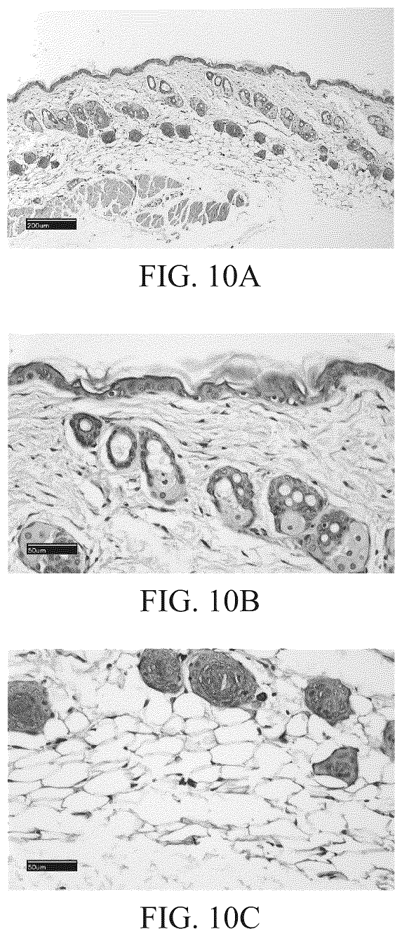

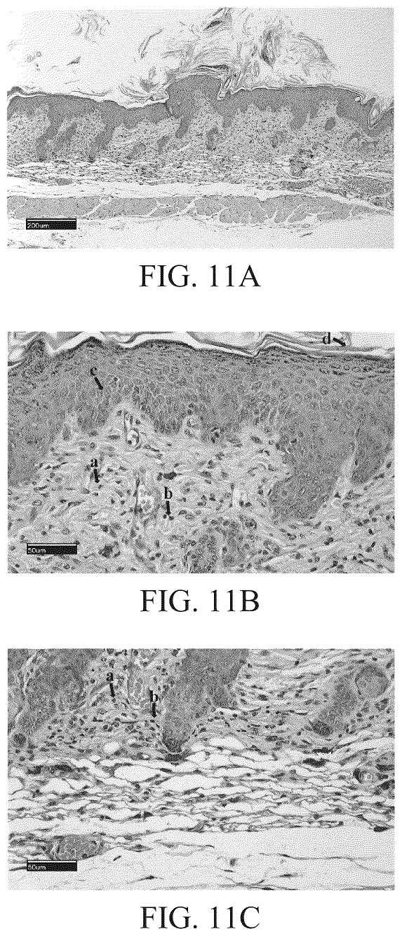

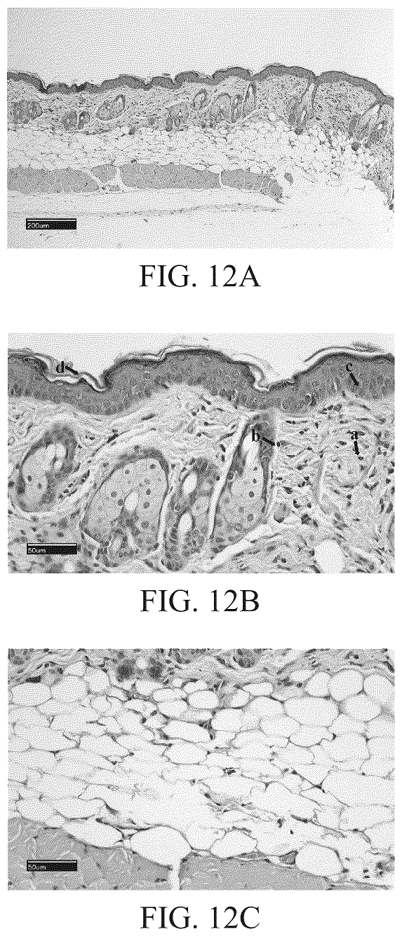

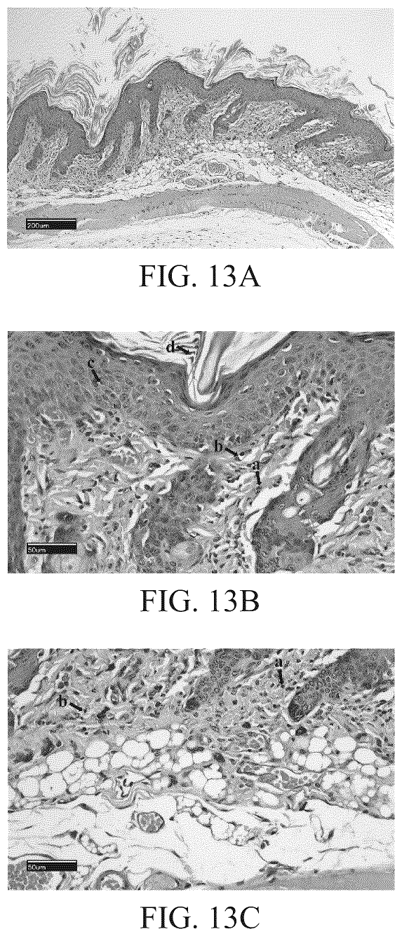

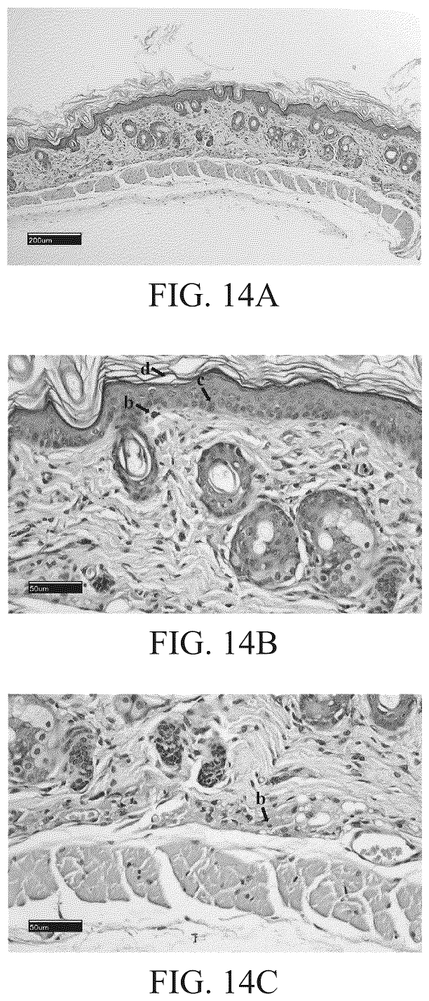

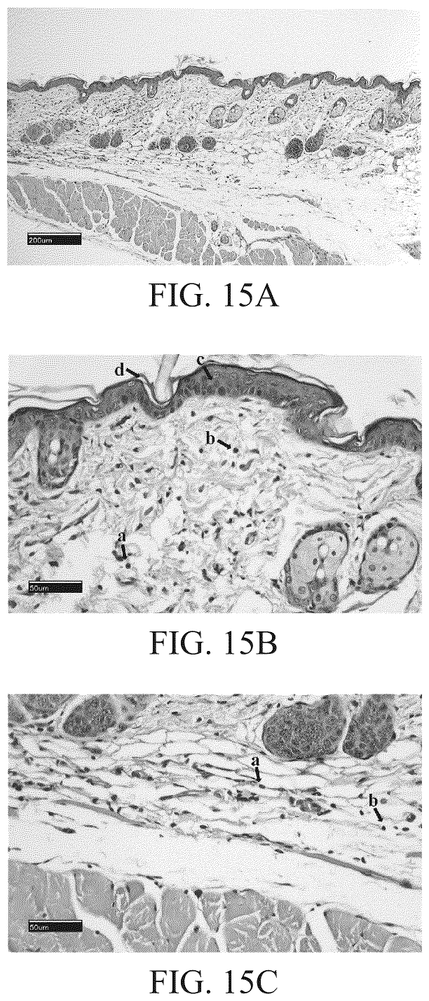

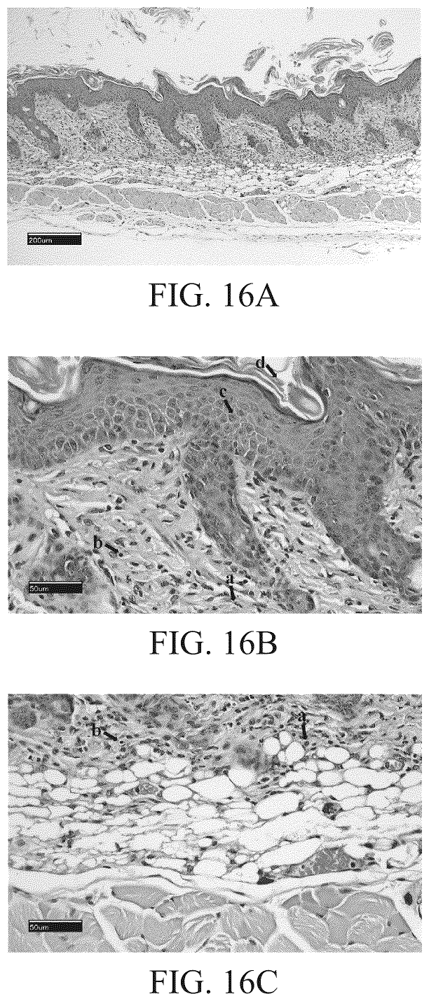

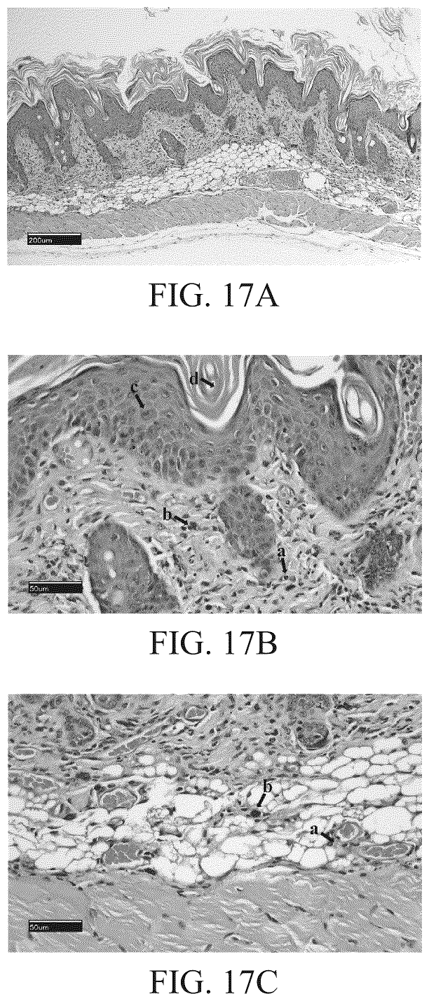

The efficacy of p-TSA can be further observed between the H&E staining of skin of different groups. As shown in FIGS. 10A-10C, Group VC (Vaseline) showed no significant histopathological change in the skin. As shown in FIGS. 11A-11C, Group IC (5% IMQ) showed that the skin had moderate/severe infiltration of neutrophils (arrow a) and mononuclear cells (arrow b) in the dermis and subcutis, moderate hyperplasia (arrow c) of epidermis and follicular epithelium, and moderate hyperkeratosis (arrow d) of epidermis. As shown in FIGS. 12A-12C, Group HDC (Vaseline+100% DMSO) showed that the skin had slight infiltration of neutrophils (arrow a) and mononuclear cells (arrow b) in the dermis and subcutis, slight hyperplasia (arrow c) of epidermis and follicular epithelium, and minimal hyerkeratosis (arrow d) of epidermis. As shown in FIGS. 13A-13C, Group IHD (5% IMQ+100% DMSO) showed that the skin had moderate/severe infiltration of neutrophils (arrow a) and mononuclear cells (arrow b) in the dermis and subcutis, moderate hyperplasia (arrow c) of epidermis and follicular epithelium, and moderate hyperkeratosis (arrow d) of epidermis. As shown in FIGS. 14A-14C, Group 14MP (5% IMQ+100% DMSO+4M p-TSA) showed that the skin had moderate infiltration of neutrophils and mononuclear cells (arrow b) in the dermis and subcutis, slight hyperplasia (arrow c) of epidermis and follicular epithelium, and slight hyperkeratosis (arrow d) of epidermis. As shown in FIGS. 15A-15C, Group LDC (Vaseline+70% DMSO) showed that the skin had slight infiltration of neutrophils (arrow a) and mononuclear cells (arrow b) in the dermis and subcutis, minimal hyperplasia (arrow c) of epidermis and follicular epithelium, and minimal hyperkeratosis (arrow d) of epidermis. As shown in FIGS. 16A-16C, Group ILD (5% IMQ+70% DMSO) showed that the skin had moderate/severe infiltration of neutrophils (arrow a) and mononuclear cells (arrow b) in the dermis and subcutis, moderate hyperplasia (arrow c) of epidermis and follicular epithelium, and slight hyperkeratosis (arrow d) of epidermis. As shown in FIGS. 17A-17C, Group 11.6MP (5% IMQ+70% DMSO+1.6 Mp-TSA) shows the skin presented moderate/severe infiltration of neutrophils (arrow a) and mononuclear cells (arrow b) in the dermis and subcutis, moderate hyperplasia (arrow c) of epidermis and follicular epithelium, and moderate hyperkeratosis (arrow d) of epidermis. Again a dose dependent p-TSA treatment effect was observed, with a larger reduction in the infiltration of neutrophils and mononuclear cells in the dermis and subcutis, hyperplasia of epidermis and follicular epithelium, and hyperkeratosis of epidermis between Group I4MP (4M p-TSA) and Group IHD as compared to between Group I1.6MP (1.6M p-TSA) and Group ILD.

The results indicated that 4M and 1.65M p-TSA showed significant therapeutic effects in treating imiquimod-induced psoriasis.

### Example 7: Use of an Animal Model to Assess Efficacy of p-TSA in Atopic Dermatitis-Like Symptoms

### Material and Methods

### 1. Animals, Induction of Atopic Dermatitis (AD)-Like Symptoms, and Treatment

a. Mouse Entry

To induce AD-like cutaneous condition in mouse, cutaneous DNCB sensitization and challenging in BALB/c mice was used. Twenty female BALB/c mice at six-weeks of age were obtained from BioLASCO, Taipei, Taiwan. Five mice each cage are housed in a controlled environment and allowed to acclimatize before experimental use. After 1 week of acclimation period, body weight of the mice was measured the day before the experiment and the dorsal area of mice was shaved and depilated.

b. Induction Method

Mice are then placed in an anesthetic chamber and anesthetized using a mixture of air and 2% isoflurane at a rate of 1 Liter per minute. Once unconscious, the mice are transferred to a stereotaxic frame, where anesthesia is maintained through a mask. The hair on the back is shaved and a thin layer of depilatory cream are applied to the shaved area for 2 minutes. The cream and remaining of hair are wiped off with damped tissue paper. This is day-8.

c. Mode of Administration

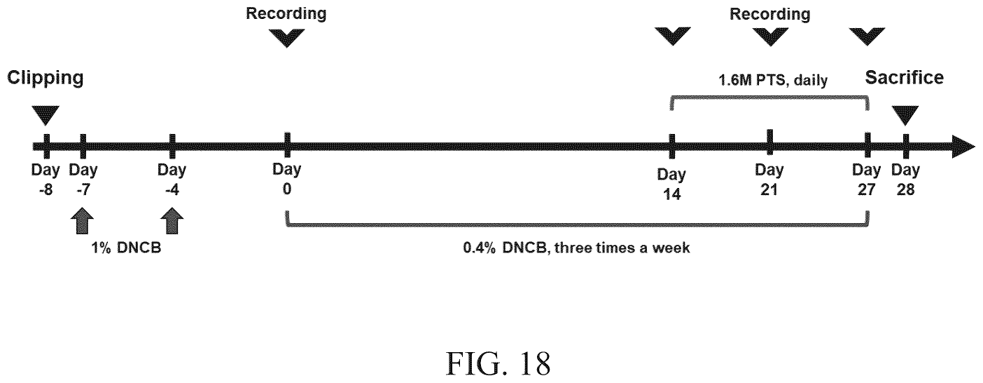

The detailed experiment time-course is shown in FIG. 18. At Day-7 and Day-4 after shaving and depilation (about 5 cm²), 200 μl 1% DNCB dissolved in an acetone:olive mixture (3:1 vol/vol) was applied on the dorsal skin of the mice (i.e., cutaneous DNCB sensitization). The DNCB-sensitized BALB/c mice were divided into four groups, see Table 10: (1) Acetone+Olive oil (3:1); (2) DNCB-induced atopic dermatitis+water; (3) DNCB-induced atopic dermatitis+vehicle; and (4) DNCB-induced atopic dermatitis+topical 25% p-TSA 1600 mM. To induce AD-like phenotypes, DNCB was applied topically on the mice three times a week in Groups 2, 3, and 4. In Group 4, p-TSA 1.6M was topically applied to the mouse dorsal skin daily for 2 weeks (Day 14-27). See Table 11 for ingredients used for the experiment and Tables 12 for formulations of the aforementioned vehicle and 25% p-TSA 1600 mM.

TABLE 10
Treatment groups for Evaluation of p-TSA in Treating
DNCB-induced Atopic Dermatitis-like Symptoms

| Group | Topic Application | Treatment | Concentration | N |
|---|---|---|---|---|
| 1 | Acetone + olive oil (3:1) | — | — | 5 |
| 2 | DNCB | Water | — | 5 |
| 3 | DNCB | Vehicle | — | 5 |
| 4 | DNCB | 25% p-TSA | 1600 mM | 5 |

TABLE 11
Ingredientsused for induction of Atopic Dermatitis
(AD)-like symptoms and treatment

| Ingredient | Use | Cas. Number | Manufacture |
|---|---|---|---|
| Para- | Experimental | 70-55-3 | Sigma Aldrich |
| toluenesulfonamide | group | | |
| 2,4- | Induce of AD | 97-00-7 | Sigma Aldrich |
| Dinitrochlorobenzene, | | | |
| DNCB | | | |
| Acetone | DNCB solvent | 67-64-1 | J. T. Baker |
| Olive Oil | DNCB solvent | — | — |
| PEG 400 | p-TSA solvent | 25322-68-3 | Sigma Aldrich |
| DMSO | p-TSA solvent | 67-68-5 | J. T. Baker |

TABLE 12
p-TSA and Vehicle Formulation

| Ingredients | | | | |
|---|---|---|---|---|
| (% w/w) | 25% p-TSA | Vehicle | Cas. number | Manufacture |
| p-TSA | 25% | 0% | 70-55-3 | Sigma Aldrich |
| PEG 400 | 22% | 22% | 25322-68-3 | Sigma Aldrich |
| DMSO | 40% | 40% | 67-68-5 | J. T. Baker |
| Pure Water | 13% | 38% | — | — |

In Group 1 as a control group, mice were observed until the end of experiment (Day 27) without any treatment. In Group 2-4, 200 μL 0.4% DNCB in an acetone:olive oil mixture (3:1 vol/vol) was applied to the dorsum three times a week for 4 weeks (day 0-27) (cutaneous DNCB challenging). 25% 1600 mM p-TSA was topically applied to dorsum (200 μL/mouse/day) in Group 4 daily for 2 weeks (Day 14-27).

d. Data Collection

Body weight: measure and record the body weight every week.

Epidermal thickness: the dorsal skin of the mice is divided into four quadrants and measured using a caliper

Dermatitis score: quantified based on erythema (redness), edema (papulation), excoriation, and desquamation (scaling). Each sign is assigned an intensity score from 0 to 3, with 0 being absent; 1, mild; 2, moderate; and 3, severe.

Duration and number of scratching: duration and number of scratching of the body with their hind paws by the recorded video of the mice was video recorded for 10 min at Day 0, 13, 21 and 27.

e. Data Analysis

Photographs are taken every day to record the disease severity including dermatitis score, duration and number of scratching

At Day 28, mice were anesthetized and sacrificed to obtain sample of dorsal skin and serum. Excised dorsal cutaneous tissue is fixed in 10% formalin for 48 hours and samples are then stained and submitted for histopathological examination by a veterinarian.

Determination of therapeutic effect is based on the calculated difference in the epidermal thickness of the DNCB-induced Atopic Dermatitis-like groups of mice on the day of sacrifice.

Scratching behavior is based on the number of scratching and duration of scratching in 10 minutes of video recording.

### Result: P-TSA can Mitigate DNCB-Induced AD-Like Skin Inflammation

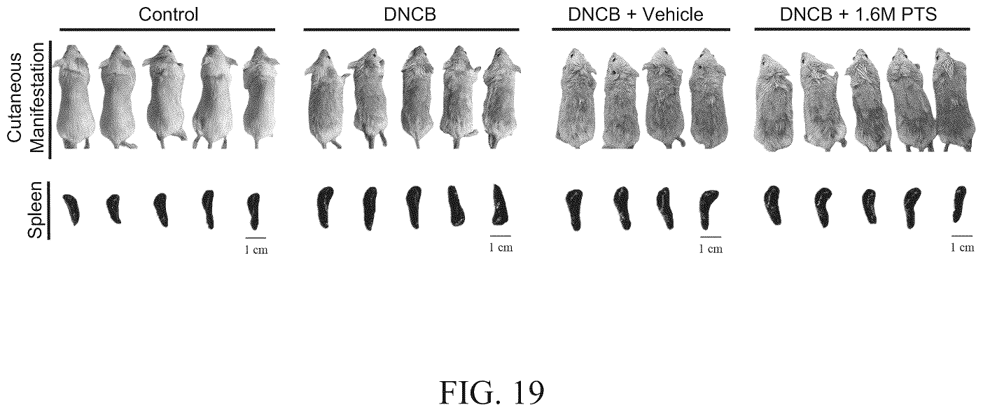

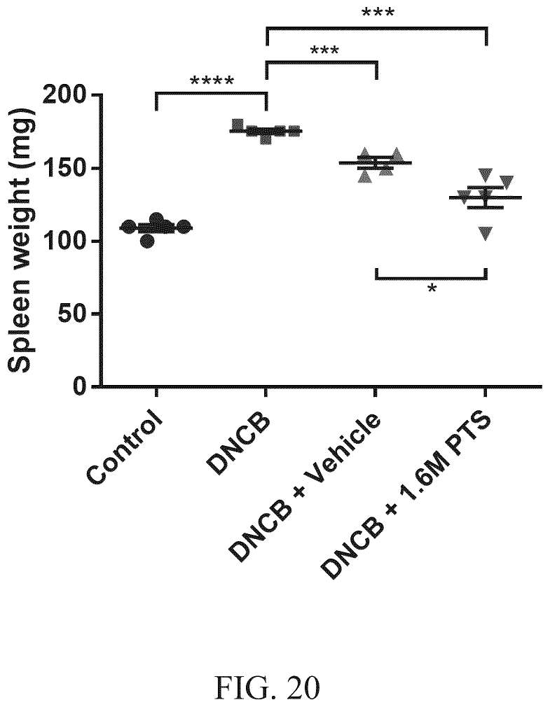

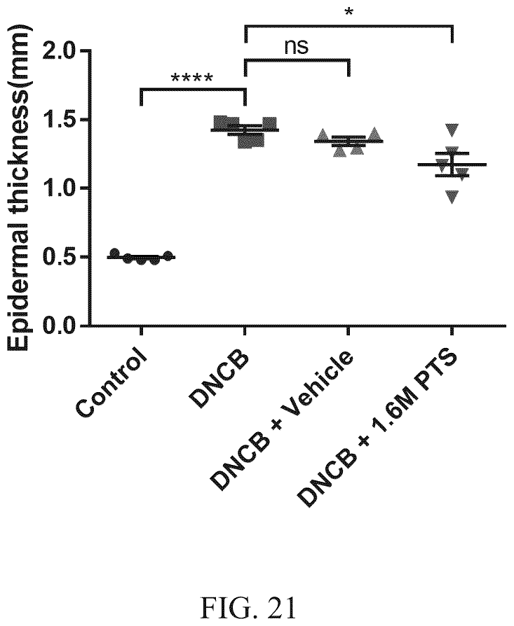

The treatment outcome of different treatment groups is presented in FIG. 19. The upper panel of FIG. 19 showed the level of cutaneous manifestations in each treatment group, where DCNV group and DCNV+Vehicle group showed cutaneous manifestations as compared to Control group, while DNCB+1.6M PTS group showed reduced cutaneous manifestations as compared to DCNV group and DCNV+Vehicle group. Further, the lower panel of FIG. 19 showed spleen of each group. Dermatitis often causes inflammation and swelling of the spleen, and therefore spleen size is a good indicator for the severity of dermatitis. Spleen size and cutaneous manifestations were further quantified in FIGS. 20 and 21, respectively. In FIG. 20, spleen weight of each treated mice were measured. The result showed that the spleen weight in the order of heaviest to lightest were DCNV group, DCNV+Vehicle group, DNCB+1.6M PTS group and Control group. Moreover, DNCB+1.6M PTS group showed a significant reduction in spleen weight as compared to both DCNV group and DCNV+Vehicle group. In FIG. 21, cutaneous manifestations were quantified using epidermal thickness. The result also showed that the epidermal thickness in the order of thickest to thinness were DCNV group, DCNV+Vehicle group, DNCB+1.6M PTS group and Control group.

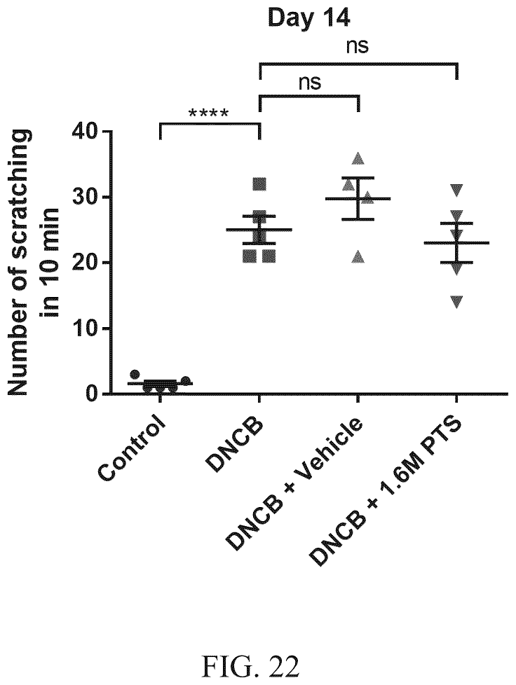

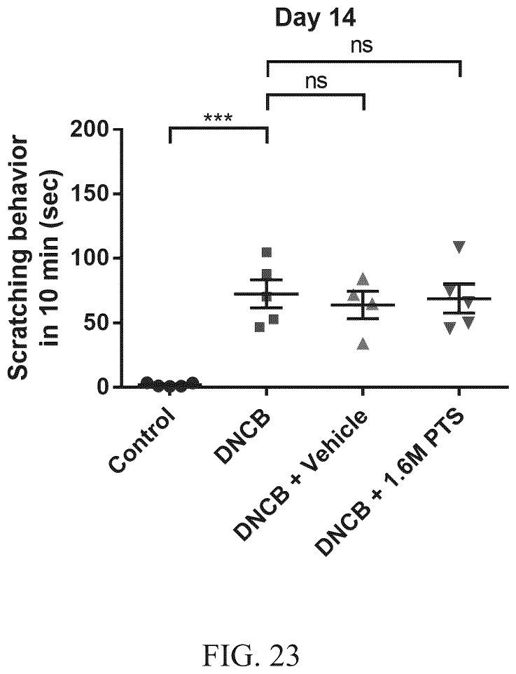

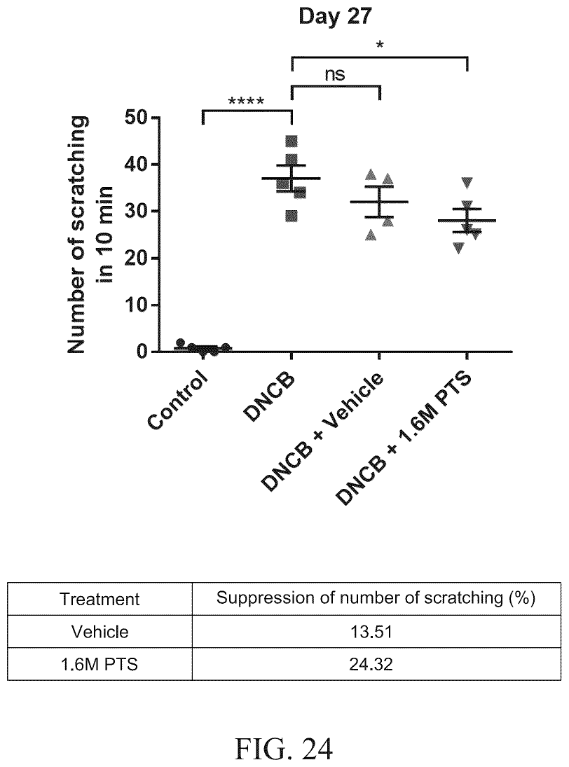

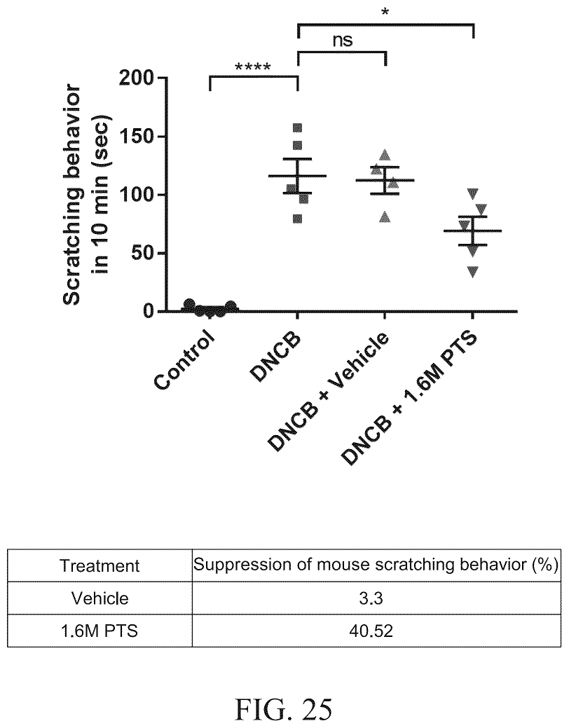

Apart from the morphological and physiological changes mentioned above, behavior changes between different treatment groups were also recorded. DNCB sensitization caused atopic dermatitis and itching, resulting in an observable scratching behavior. As shown in FIGS. 22 and 23, before mitigation and treatment of DNCB-induced dermatitis (i.e., PTS treatment), DCNV group, DCNV+Vehicle group and DNCB+1.6M PTS group had similar number and duration of scratching, and were all significantly more than that of Control group. As shown in FIGS. 24 and 25, after mitigation and treatment of DNCB-induced dermatitis for 2 weeks, DNCB+1.6M PTS group had the least number and duration of scratching in the three DNCB-induced dermatitis induced group. Furthermore, the changes in scratching behavior after sham treatment (DCNV+Vehicle group) and PTS treatment (DNCB+1.6M PTS group) were: 1) suppression of number of scratching was 13.51% in sham treatment and 24.32% in PTS treatment; and 2) suppression of mouse scratching behavior was 3.3% in sham treatment and 40.52% in PTS treatment. Therefore, treatment with 1.6M PTS successfully treated itching and reduced scratching.

Based on the above results, DNCB induced thickening of the epidermis, enlargement of the spleen, and more scratching behavior, all of which were indicative of Atopic Dermatitis. Treating mice having DNCB-induced dermatitis with PTS significantly mitigate the aforementioned parameters, thereby demonstrated PTS ability to treat Atopic Dermatitis.

### REFERENCE

Jang, S., Ohn, J., Kim, J. W. et al. Caffeoyl-Pro-His amide relieve DNCB-Induced Atopic Dermatitis-Like phenotypes in BALB/c mice. Sci Rep 10, 8417 (2020).
Alyoussef A. Arjunolic acid protects against DNCB-induced atopic dermatitis-like symptoms in mice by restoring a normal cytokine balance. Eur Cytokine Netw. 2015 April-June; 26 (2): 38-45. doi: 10.1684/ecn.2015.0364. PMID: 26553587.

What is claimed is:

1. A method for preventing or treating an inflammatory skin disease, comprising administering an effective amount of a composition to a subject in need thereof, wherein the composition comprises a benzenesulfonamide derivative and a pharmaceutically or cosmetically acceptable excipient thereof.

2. The method according to claim 1, wherein the benzenesulfonamide derivative is represented by formula (I): 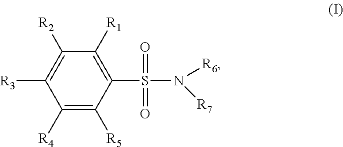
or a pharmaceutically acceptable salt thereof,
wherein R₁ to R₇ are independently selected from the group consisting of H, a C₁-C₆ linear or branched alkyl group, a C₁-C₆ linear or branched alkoxy group, a C₃-C₆ cycloalkyl group, a C₃-C₆ cycloheteroalkyl group, an amino group, and a halo group, or R₆ and R₇ are linked to each other to form a ring,
wherein the alkyl group, the alkoxy group, the cycloalkyl group, the cycloheteroalkyl group, and the ring in R₁ to R₇ are independently unsubstituted or substituted with one or more substituents.

3. The method according to claim 2, wherein the substituent is selected from the group consisting of phenyl, halo, oxo, ether, hydroxyl, carboxyl, amino, sulfo, and sulfonamide groups.

4. The method according to claim 3, wherein the benzenesulfonamide derivative is selected from the group consisting of para-toluene sulfonamide, ortho-toluene sulfonamide, meta-toluene sulfonamide, N-ethyl-para-toluene sulfonamide, N-ethyl-ortho-toluene sulfonamide, and N-cyclohexyl-para-toluene sulfonamide.

5. The method according to claim 1, wherein the inflammatory skin disease is selected from the group consisting of herpes simplex, shingles, eczema, psoriasis, urticaria, and any combination thereof.

6. The method of claim 5, wherein the inflammatory skin disease is atopic dermatitis.

7. The method of claim 1, wherein the composition agent is administered to the subject topically, orally, intravenously, subcutaneously, intradermally, orally, intrathecally, intraperitoneally, intranasally, intramuscularly, intrapleuraly, topically, or through nebulization.

8. The method of claim 1, wherein the composition agent is administered to the subject topically.

9. The method according to claim 1, wherein the pharmaceutically or cosmetically acceptable excipient is selected from the group consisting of a preservative, a lubricant, a suspending agent, a wetting agent, a flavoring agent, a thickening agent, a biocompatible solvent, a surfactant, a complexation agent, and any combination thereof.

10. The method according to claim 9, wherein the biocompatible solvent is selected from the group consisting of polyethylene glycol, propylene glycol, glycerol, sorbitol, ethanol, dimethyl sulfoxide, N-methyl-2-pyrrolidone, N,N-dimethylacetamide, glycofurol, acetone, isopropyl alcohol, triglyceride, benzyl benzoate, benzyl alcohol, solketal, and any combination thereof.

11. The method according to claim 9, wherein the surfactant is selected from the group consisting of lecithin, macrogol 15 hydroxystearate, polyoxyethylene alkyl ether, polyoxyethylene castor oil, polyoxyethylene sorbitan fatty acid ester, polyoxyethylene stearate, polyoxylglyceride, sorbitan ester, tocopheryl polyethylene glycol succinate, and any combination thereof.

12. The method according to claim 9, wherein the complexation agent is polyvinyl pyrrolidone or cyclodextrin.

13. The method according to claim 1, wherein the benzenesulfonamide derivative is present in an amount ranging from about 1% to about 70% by weight.

14. The method according to claim 1, wherein the pharmaceutically or cosmetically acceptable excipient is present in an amount ranging from about 30% to about 99% by weight.

15. The method according to claim 1, wherein the composition is in a form suitable for topical administration.

16. The method according to claim 15, wherein the composition is in a form of a gel, a soft gel, a lotion, an ointment, a paste, an emulsion, a cream, a slurry, a patch, a film, a cream, or an aerosol.

17. The method according to claim 1, wherein the composition is administered to the subject 1 time to 70 time a week.

18. The method according to claim 1, wherein the composition is administered to the subject 1 time to 10 time a day.

19. The method according to claim 1, wherein the composition is continuously administered to the subject for at least 1 week.

20. The method according to claim 19, wherein the composition is continuously administered to the subject for at least 2 weeks.
